(19) United States
(12) Patent Application Publication
(10) Pub. No.: US 2011/0087171 A1
(43) Pub. Date: Apr. 14, 2011

(54) METHOD AND APPARATUS TO SECURE LIFE SUPPORT LINES TO AN INFANT DURING NEONATAL CARE

(76) Inventor: Jennifer Bracci, Miami, FL (US)

(21) Appl. No.: 12/578,024

(22) Filed: Oct. 13, 2009

(51) Int. Cl. A61M 5/00

(52) U.S. Cl. 604/174

(57) ABSTRACT

This invention is directed to a method for affixing an apparatus to a parent to allow skin-to-skin contact with an infant connected to medical tubing. The method includes placement of medical tubes and/or sensors into a two-part clip on the apparatus having a first clip portion and a corresponding second clip portion—both connected through a spring action hinge. A locking means on the first clip portion next affixes to the second clip portion to ensure the medical tubing does not become dislodged. After ensuring there are no kinks or bends in the medical tubing, a fastening means (for example, a flat panel with an adhesive layer) attached to the apparatus is positioned proximate to the parent's chest (or article of clothing) and then affixed. A rotating means (between the two-part clip and fastening means) can be used to rotate the apparatus to ensure the comfort of the neonatal infant.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

This invention relates to an apparatus secured to the parent of a recently birthed low weight infant in order to secure and maintain a plurality of life support lines and respiratory interfaces connected to the infant during kangaroo care provided in a Neonatal Intensive Care Unit (NICU).

### BACKGROUND OF THE INVENTION

In most modern cultures, the act of birthing has become a medical procedure performed in a hospital or clinic. Once delivered, the infant is treated by health professionals who provide care apart and away from the mother. Often, infants are washed, weighed, measured and nourished by these healthcare professionals. However, studies have shown that these practices (however highly important to the overall health of the child) can be physiologically and emotionally disturbing to the infant, who has been securely within and in constant contact with the mother and become accustomed to her rhythms and physiology.

When the infant is born premature or underweight, there exist additional concerns and stressors regarding separation of the infant with the mother. These premature or underweight infants are often placed in neonatal intensive care nurseries (NICUs) within the hospital that provide specialized and direct care to these infants (hereinafter “neonatal infants”). While often necessary to maintain the health of the child, these NICU facilities unfortunately offer an unfamiliar and sterile environment for neonatal infants.

Neonatal infants are maintained in self-contained clear incubators that include thermostats to keep the infant warm and visible. Often, they are connected to a variety of life support and respiratory systems, as their organs are not fully developed. These can include an endotracheal tube, a continuous positive-airway pressure (CPAP) interface to assist in breathing, a pic line, and/or an intravenous fluid (IV) line (collectively, “medical tubing”). The neonatal infant is also attached to a series of heartbeat, oxygen and body temperature monitors (collectively, “medical sensors”). Placement in incubators within the NICU risks multiple side-effects as they further remove the neonatal infant from the mother for longer periods of time.

A medical technique commonly referred to as “kangaroo care” has provided one viable option to restore closeness of a neonatal infant with his or her mother by placing the infant in skin-to-skin contact with a parent (often, but not necessarily always, the mother). Essentially, the neonatal infant is temporarily removed from the incubator within the NICU and placed on the bare chest of the mother. This practice ensures physiological and psychological warmth and bonding. The mother's body responds to the needs of the infant directly, helping regulate temperature more smoothly than an incubator, her milk adjusts to the nutritional and immunological needs of her fragile infant, and the baby sleeps more soundly.

When practicing “kangaroo care,” the neonatal infant only wears a diaper and is positioned proximate to the parent's chest. The mother wears a shirt or hospital gown having an opening in the front to allow skin-to-skin contact.

The kangaroo method can be used continuously around the clock or for shorter periods per day gradually increasing as tolerated by the neonatal infant. The practice preferably begins several days or even weeks after birth depending upon the health of the infant. It is preferable to conduct kangraroo care for about six weeks to establish routine breastfeeding and physiological recovery from the birth process.

Over 200 NICUs throughout the United States (and over 80% of hospitals) currently offer and promote kangaroo care for neonatal infants. However, providing kangraroo care to neonatal infants that require life support and respiratory assistance often presents logistical dilemmas. When removing the neonatal infant from the incubator for placement on the mother's chest, the practice requires multiple NICU nurses and related staff to transfer all of the various medical tubes and sensors. Unfortunately, movement of the child and/or mother during kangaroo care can risk removal, extubation and/or displacement of these life support and respiratory systems. While often not life threatening, this can cause discomfort and injury to the neonatal infant.

Very little has been done to prevent the exubation and/or displacement of life support during kangaroo care. Often NICU nurses and related staff will simply tape life support onto the parent. While somewhat effective in temporarily maintaining these tubes, the plurality of tape must be removed when the child returns to the incubator, often causing discomfort to the mother. Others have developed complicated vests that have a plurality of hook-and-loop fasteners to affix the lifesupport tubing to the mother. One example of this is shown in United States Patent Application No. 2006/0206978 to Hilton et. al. However, the bulky vest taught by Hilton has various drawbacks. First, Hilton's vest makes it more difficult to achieve actual skin-to-skin contact. Second, it requires the mother to dawn the vest before kangaroo care and then remove the vest afterwards—all of which takes time away from the actual care to the neonatal infant. Third, the use of hook-and-loop fasteners does not necessarily prevent occlusion or twisting of the various life-support and respiratory systems, as any movement may cause a kink in the tubing.

Accordingly, there is a need in the art of kangaroo care for a simple yet effective device for the mother to wear when offering skin-to-skin contact that reduces the risk of extubation and/or displacement of life support and respiratory systems. Moreover, such a device should further eliminate the risk of twisting or reducing the flow of life support during kangaroo care. The device should preferably be disposable, capable of being maintained in a sanitary state, easily placed and/or affixed on the mother, and allows for quick transfer of the neonatal infant from the incubator to the mother.

### SUMMARY OF THE INVENTION

In view of the foregoing background, this invention solves the limitations found in current methods (and related devices) for affixing medical tubing and sensors to a parent when performing kangaroo care with a neonatal infant connected to life support and/or respiratory systems. The preferred method first includes placement of medical tubes and/or sensors within a two-part clip (having a first clip portion and a corresponding second clip portion which connect to one another through a first spring action hinge). This is accomplished by contacting medical tubes and/or sensors on the first clip portion and rotating the second clip portion (through use of the first spring action hinge) onto the medical tubes and/or sensors. In addition, a locking means attached to the first clip portion can be affixed to the second clip portion to ensure the medical tubes and/or sensors do not become dislodged.

Once both clip portions are locked, the method contemplates ensuring that the medical tubes and/or sensors are not occluded or bent. Finally, a fastening means attached to the apparatus is positioned proximate to the parent's chest (or article of clothing) and then affixed. The method can further comprise the step of rotating the two-part clip (in relation to the fastening means) through use of a rotating means to ensure the comfort of the neonatal infant.

In addition to the preferred method described above, the invention is further directed to an apparatus. Apart from the apparatus discussed in reference to the preferred method, the two-part clip can further include a compressible material to prevent the medical tubing and/or sensors from occluding. More specifically, the first clip portion can also include a compressible material on its bottom side, while the second clip portion has a similar compressible material located on its top side. The first spring axis hinge is attached to a rotatable member, which in turn is connected to the top side of a flat panel. At the bottom side of the flat panel is an adhesive layer sufficient to affix the flat panel to a parent.

As an alternative to the flat panel that includes an adhesive layer, a second embodiment contemplates use of a pair of jaws pivotally mounted on a second spring axis hinge sufficient to attach an article of clothing worn by the parent. In this second embodiment, the jaws are connected to the first spring axis hinge (either directly or indirectly). Again, the apparatus can also include a rotating member between the pair of jaws and the first spring axis hinge to help position the first clip portion and second clip portion to effectively hold and maintain the medical tubing and sensors.

### BRIEF DESCRIPTION OF THE DRAWINGS

FIG. 1 is a front view of the apparatus that includes a flat panel with an adhesive layer.

FIG. 2 is a top view of the apparatus shown in FIG. 1.

FIG. 3 illustrates attachment of the apparatus shown in FIG. 1 on a parent to allow skin-to-skin contact.

FIG. 4 is a front view of the apparatus that includes a pair of jaws that connect to an article of clothing worn by a parent.

FIG. 5 is a top view of the apparatus shown in FIG. 4.

FIG. 6 illustrates attachment of the apparatus shown in FIG. 4 on a parent to allow skin-to-skin contact.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

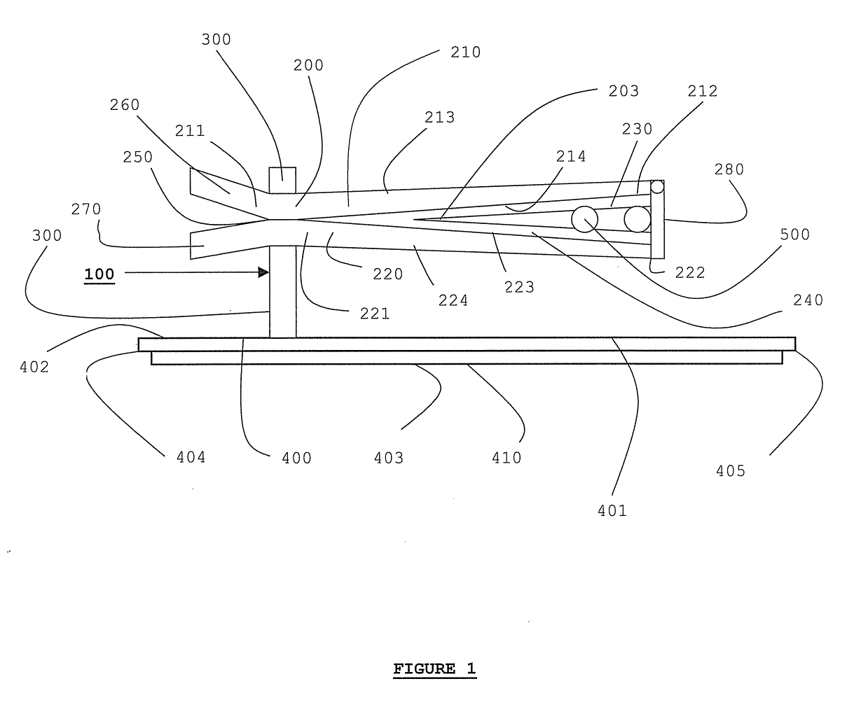

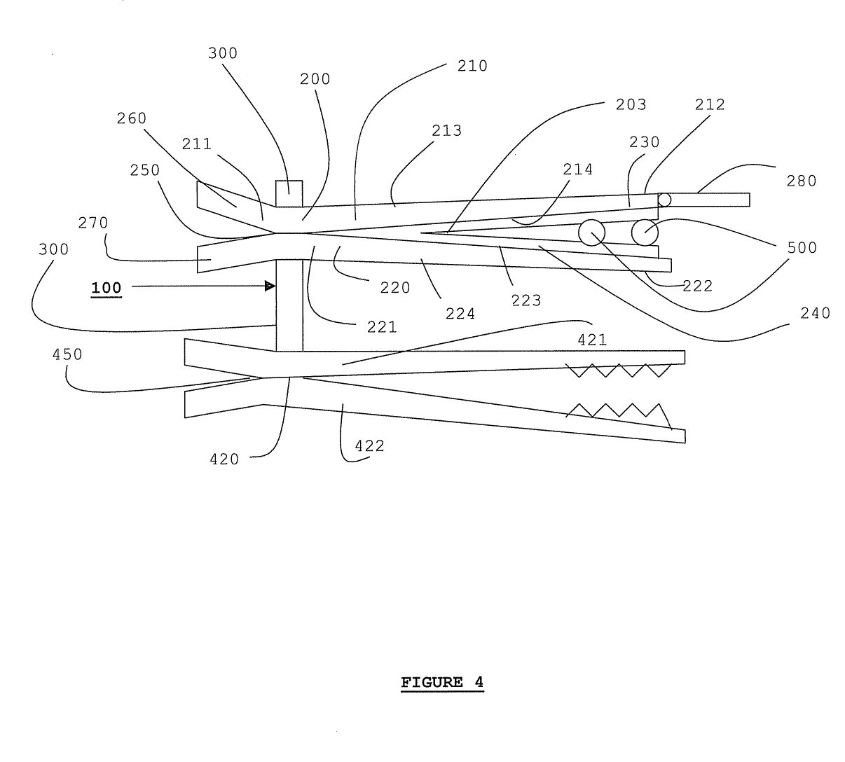

One preferred embodiment of the invention is illustrated in FIGS. 1 through 3, which teach an apparatus 100 that comprises a two-part clip 200 (that includes a first clip portion 210 attached to a corresponding second clip portion 220), and a rotatable member 300 in turn affixed to a fastening means 400. The fastening means 400 taught in FIGS. 1 through 3, described in greater detail below, is an essentially rectangular flat panel 401 that includes a top side 402 and bottom side 403, wherein an adhesive 410 is affixed to the bottom side 403 of the flat panel 401. An alternative embodiment is shown in FIGS. 4 through 6, wherein the fastening means 400 is a pair of jaws 420 having a first jaw member 421 attached (via a second spring action hinge 450) to a corresponding second jaw member 422 which connects to an article of clothing 701 worn by a parent 700. Either apparatus 100 should be capable of holding medical tubing and sensors 500 connected to a neonatal infant 600.

### The Two-Part Clip and Rotatable Member

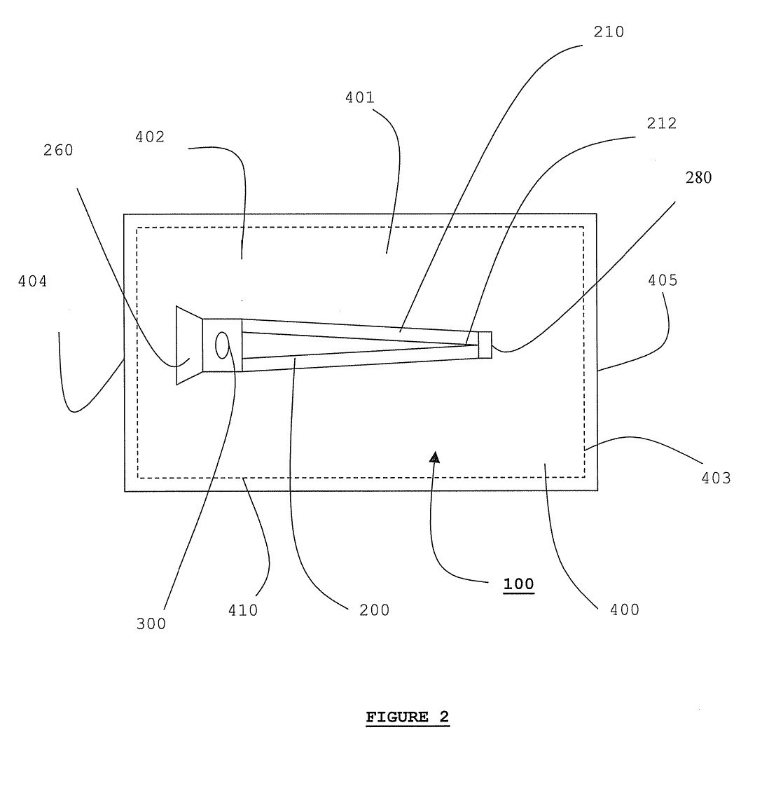

FIG. 1 provides a side view of one preferred embodiment of the apparatus 100, which includes a two-part clip 200. Although a variety of shapes and orientations can be employed for the two-part clip 200, the device should nonetheless be capable of holding a plurality of medical tubing and sensors 500 without risk of occluding or causing a kink in the life support and respiratory systems. While the two-part clip 200 (further shown in FIG. 2) is preferably made of a light-weight and sturdy plastic, any similar material (including a metal and/or composite) can be used. Moreover, any similar material capable of being maintained in a sanitary state, known to those of ordinary skill in the art, can be used.

As illustrated in FIG. 1, the two-part clip 200 includes a first clip portion 210 having a first end 211, a corresponding second end 212, a top side 213 and a corresponding bottom side 214. The first clip portion 210 is essentially rectangular or square in shape, but can be designed in any shape that allows support for a plurality of medical tubing and sensors 500.

Preferably affixed on the bottom side 214 of the first clip portion 210 is a first compressible sheet 230. The compressible sheet 230 is essentially flat and rectangular in orientation and can be manufactured from any foam-like material. Moreover, the compressible sheet 230 can be made of neoprene or a similar spongy material known to those of ordinary skill in the art.

In addition to the first clip portion 210, FIG. 1 shows the two-part clip 200 further includes a corresponding second clip portion 220. The second clip portion 220 includes a first end 221, a corresponding second end 222, a top side 223 and a corresponding bottom side 224. The size and dimensions of the second clip portion 220 should mirror those of the first clip portion 210. Accordingly, the distance between the first end 221 and second end 222 of the second clip portion 220 should be roughly the same as the distance between the first end 211 and second end 212 of the first end portion 220. Again, the second clip portion 220 is essentially rectangular or square in shape, but can be designed in any shape that allows support for a plurality of medical tubing and sensors 500.

Affixed to the top side 223 of the second clip portion 220 is a second compressible sheet 240. Again, the compressible sheet 240 is essentially flat and rectangular in orientation and can be manufactured from any foam-like material. Moreover, when the medical tubing and sensors 500 are placed between both compressible sheets 230 and 240, they are maintained and held without risk of occluding or kinking any of the respiratory systems which may risk providing the neonatal infant 600 with needed oxygen and nutrients. Preferably, a locking mechanism 280 is attached to either the first clip portion 210 or second clip portion 220 (at or proximate to their second ends 212 and 222), which can affix to (and/or latch onto) the corresponding clip portion (220 or 210) in order to prevent the various medical tubing and sensors 500 from slipping out of the two-part clip 200.

As further shown in FIG. 1, a first spring action hinge 250 connects the first clip portion 210 to the second clip portion 220. The first spring action hinge 250 can ensure a downward compression force (constricting both clip portions 210 and 220 together) or manufactured to create an upward compression force (pulling clips 210 and 220 apart). Should the first spring action hinge 250 be designed to create an upward compression force, the two-part clip 200 should include the aforementioned locking mechanism 280 at the distal end of both clip portions 210 and 220. In the embodiment shown in FIG. 1, the first spring action hinge 250 is positioned between both clip portions (210 and 220) and constricts both parts of the two-part clip 200 toward one another. By doing so, the spring action hinge 250 moves the first compressible sheet 230 toward the second compressible sheet 240 to surround and hold the variety of medical tubing and sensors 500. Under this embodiment where the first spring action hinge 250 constricts (rather than pulls apart) both clip portions 210 and 220, the apparatus 100 can include tabs 260 and 270 to assist in opening the two-part clip 200. More specifically, FIG. 1 illustrates how the first spring action hinge 250 can be opened by squeezing both the first tab 260 and second tab 270 located at one end of the two-part clip 200. The first tab 260 is preferably positioned at the first end 211 of the first clip portion 210, while the second tab 260 is located at the first end 221 of the second clip portion 220.

FIG. 2 offers a perspective view of the first embodiment of the apparatus 100. As shown, the apparatus 100 can further include a rotatable member 300. The rotatable member 300 can be a rivet or a similar mechanism that allows the two-part clip 200 to pivot to better maintain and position the medical tubing and sensors 500 to provide greater comfort for the neonatal infant 600. In addition, the rotatable member 300 can allow a lock-in-groove 310 that allows the two-part clip 200 to stay in a maintained orientation until released and/or repositioned during use.

### First Embodiment of the Fastening Means

FIGS. 1 and 2 further disclose one preferred embodiment for the fastening means 400. In all of the embodiments, the two-part clip 200 attaches to a fastening means 400 (either directly or through the rotatable member 300). Under the embodiment shown in FIGS. 1 and 2, the fastening means 400 is an essentially flat panel 401. This flat panel 401 is essentially square or rectangular in shape and includes a top side 402 and corresponding bottom side 403. However, the flat panel 410 can be any geometric shape, as long as it provides a sufficient amount of surface area to affix to the parent 700. Moreover, the flat panel 410 can have an ergonomic shape sufficient to mirror the curvature of a parent's chest area.

As shown in FIG. 2, the flat panel 401 includes a first edge 404, a corresponding second edge 405, a top edge 406 and a corresponding bottom edge 407. The flat panel 401 can be made of any light-weight and rigid material, but is preferably made of a plastic, resin and/or composite. However, the flat panel 401 can be made of any similar material know to those of ordinary skill in the art.

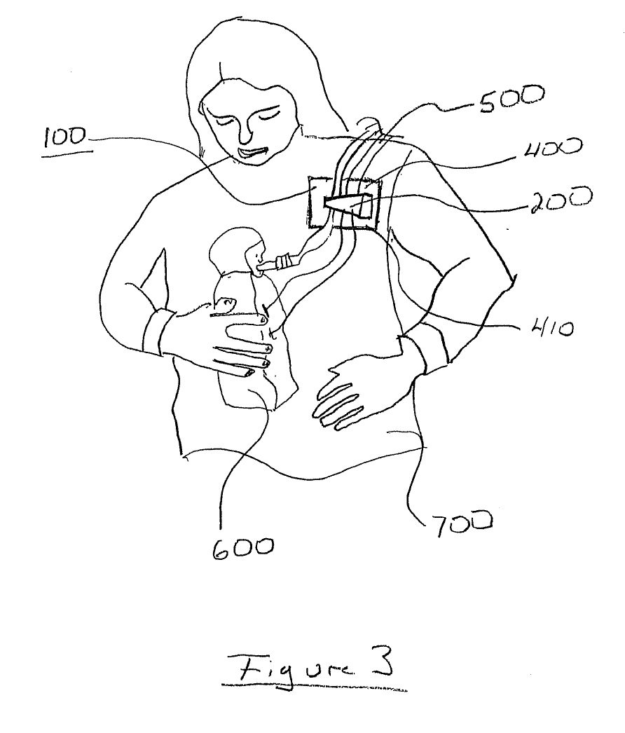

An adhesive 410 is affixed to the bottom side 403 of the flat panel 401. Any hypoallergenic material known to one of ordinary skill in the art may be used for the adhesive 410. However, it must be strong enough to allow the apparatus 100 to attach to the skin of a parent 700, and maintain all of the components (i.e., the two part clip 200, flat panel 401 and medical tubing and 500). FIG. 3 offers a direct view of the apparatus 100 affixed to the parent 700, wherein the fastening means 400 is the flat panel 410 that includes an adhesive 410.

### Second Embodiment of the Fastening Means

In addition to having a flat panel 401 as the fastening means 400, FIGS. 4 through 6 offer an alternative embodiment to help secure the apparatus 100 to the parent 700. More specifically, FIGS. 4 through 6 show how the fastening means 400 can be affixed to an article of clothing 701 (and/or strap system) worn by the parent 700. As shown in FIG. 4, this second embodiment of the fastening means 400 includes a pair of jaws 420. The pair of jaws 420 includes a first jaw member 421 and a corresponding second jaw member 422. Both jaw members 421 and 422 can include a plurality of teeth 423 that assist in affixing the jaws 420 onto an article of clothing 701.

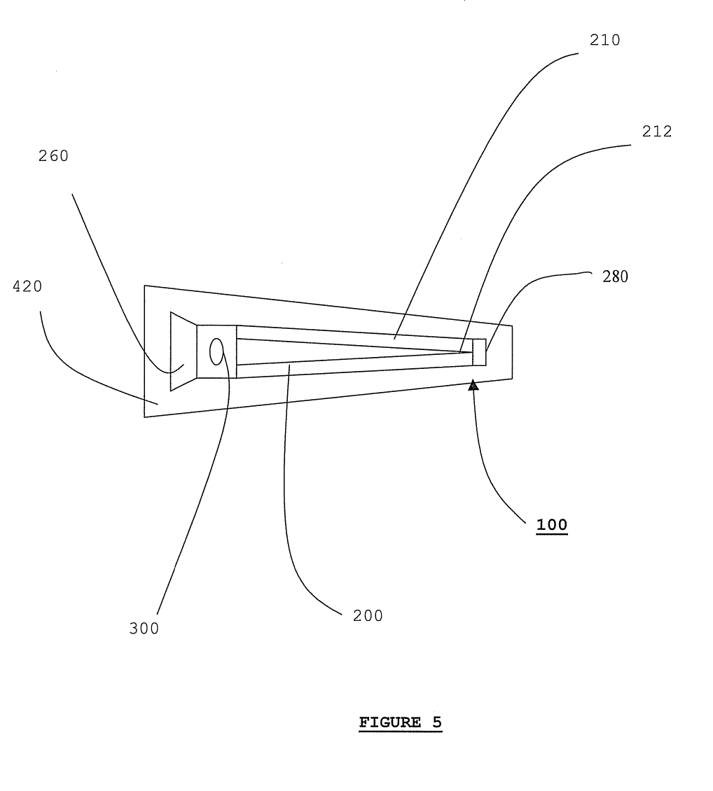

FIG. 5 provides a side view of the preferred orientation of the pair of jaws 420 employed as a fastening means 400. As illustrated in FIG. 5, both jaw members 421 and 422 of the pair of jaws 420 are preferably connected through a second spring action hinge 450. The second spring action hinge 450 is positioned between both jaw members (421 and 422) and constricts both pairs of jaws 420 toward one another.

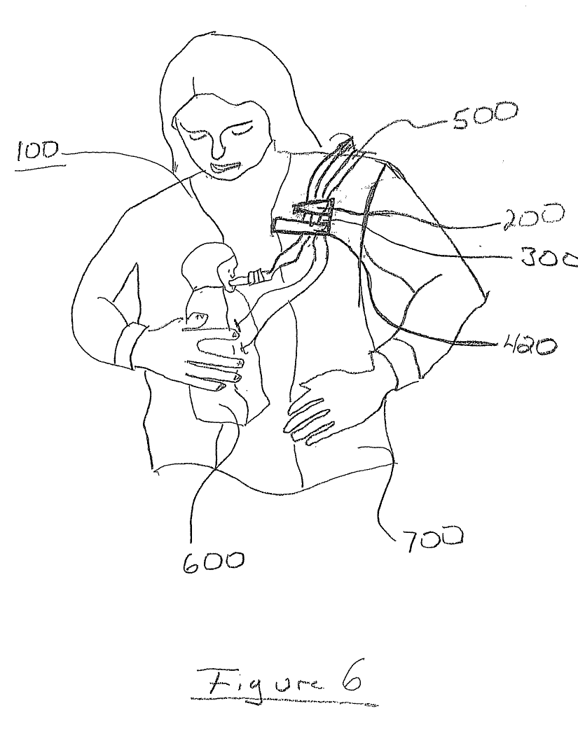

FIG. 6 shows how the pair of jaws 420 connect to a parent 700. Here, the second jaw member 422 is placed between the parent 700 and article of clothing 701. Next, the first jaw member 421 is rotated (through use of the second spring action hinge 450) onto the article of clothing 701. Through the compressing force provided by the second spring action hinge 450 the teeth 423 of the pair of jaws 420 ensures the apparatus 100 will not detach from the article of clothing 701. Once the pair of jaws 420 are affixed, the rotating member 300 (positioned between the pair of jaws 420 and the two-part clip 200) can be used to properly place the two-part clip 200 in an orientation that allows the medical tubing and sensors 500 to be properly positioned to ensure that they are not kinked or risk being removed from the neonatal infant 600. Moreover, a lock-in-groove 310 can help ensure that once positioned, the two-party clip 200 says in the same orientation until released and/or repositioned during use.

Using Multiple Apparatus in Combination

It will be understood by one of ordinary skill in the art that a plurality of apparatus 100 can be affixed to a single parent 700 when providing kangaroo care for a neonatal infant 600. For example, for a neonatal infant 600 connected to a large amount of respiratory and life support devices, two apparatus 100 can be used (one for respiratory systems such as CPAP and ventilator tubing connected to an endotracheal tube while a second for life support systems) wherein the fastening means 400 are flat panels 401). Here, both flat panels 401 can be affixed proximate to one another, and their two-part clips 200 oriented (through positioning each rotating means 300) to ensure a proper placement and maintenance of both the life support and respiratory interfaces.

In addition, multiple apparatus 100 can be used together having different fastening means 400. In one contemplated use of the invention, medical tubing 500 (ventilator tubing, CPAP, etc.) can be affixed through an apparatus 100 having a flat panel 401 as the fastening means 400, while a second apparatus 100 using a pair of jaws 420 affixes medical sensors 500 (heartbeat, temperature, oxygen monitors) to an article of clothing 701 (positioned above the flat panel 401). One of ordinary skill in the art will appreciate and recognize other similar orientations and combinations of multiple apparatus 100 to be placed on parents 700 when providing kangaroo care to a neonatal infant 500.

### Preferred Method

In addition to the embodiments of the apparatus 100 described above, the invention is further directed toward a preferred method to providing kangaroo care for neonatal infants 600 which require use of life support and respiratory systems that include medical tubing and sensors 500. The method begins by first placing the medical tubing 500 into the two-part clip 200 shown in FIGS. 1 and 4. In the embodiment where the first spring action hinge 250 creates a compression force, this is accomplished by first opening the two-part clip 200 by squeezing both the first tab 260 and second tab 270 (which in turn applies pressure to the first spring action hinge 250) to create a gap between the first clip portion 210 and the second clip portion 220. Next, medical tubing 500 is placed on the first compressible sheet 230 of the first clip portion 210. Next, the second clip portion 220 is rotated onto the medical tubes 500 through releasing the first spring action hinge 250 (by discharging both tabs 260 and 270). Upon sufficient rotation of the second clip portion 220, the second compressible sheet 240 likewise contacts the medical tubes 500 sufficient to secure these tubes without risking kinking or pinching the tubes. Next, the locking means 280 is used to block and/or cover the opening of the distal ends (212 and 222) of both clip portions 210 and 220 (as well as secure both clip portions 210 and 220 together). A visual inspection should also be conducted to ensure the medical tubes and sensors 500 are not occluded.

Should the apparatus 100 include the fastener 400 taught in FIGS. 1 through 3, the preferred method next contemplates positioning the bottom side 403 of the flat panel 401 proximate to the chest of the parent 700 (shown in FIG. 3). Next, the flat panel 401 is affixed to the parent contact with an adhesive layer 410 affixed to the flat panel 401. Upon affixing the flat panel 410 to the parent 700, the two-part clip 200 can be rotated to ensure the comfort of the neonatal infant 600 through use of the rotatable member 300.

Should the apparatus 100 instead employ the fastener shown in FIGS. 4 through 6, the method instead contemplates connecting a pair of jaws 420 onto an article of clothing worn by the parent 700. This includes first placing a first jaw member 421 proximate to one side of the article of clothing 701 and then rotating the second jaw member 422 such that the second side of the article of clothing. This is done by releasing the second spring axis hinge 450. Upon affixing the pair of jaws 420 to the parent 700, the two-part clip 200 can be rotated to ensure the comfort of the neonatal infant 600 through use of the rotatable member 300.

As discussed in greater detail above, upon affixing one apparatus 100 onto a parent 700, one or more additional apparatus 100 can next be attached directly to the parent 700 (or on an article of clothing 701 worn by that parent 700). By using the method described immediately above, a first apparatus 100 can first attached through affixing an adhesive 410 located on the bottom side 403 of the flat panel 401 onto a parent 700—to maintain medical tubing 700 (ventilator tubing, CPAP, etc.). Next, a second apparatus 100 can be attached to an article of clothing 701 through rotating a pair of jaws 420 to hold a variety of medical sensors 700 (heartbeat monitor, temperature, etc.). One of ordinary skill in the art will recognize other methods to affix other combinations of medical tubing and sensors 500 through use of multiple apparatus 100 in combination.

I claim:

1. A method of securing to a parent a plurality of medical tubing or sensors connected to an infant, comprising the steps of:
(a) placing one or more medical tubes within a two-part clip having the first clip portion and a corresponding second clip portion; said first clip portion having a top side, a bottom side, a first end and a second end;
(b) contacting the medical tubes with the first clip portion;
(c) rotating the second clip portion onto the medical tubes; said second clip portion having a top side, a bottom side, a first end and a second end;
(d) ensuring that the medical tubes are not occluded;
(e) positioning a fastening means proximate to the parent; and
(f) affixing the fastening means to either the parent or an article of clothing worn by the parent.

2. The method of claim 1, further comprising the step of:
rotating the two-part clip to ensure the comfort of an infant connected to one or more medical tubes through use of a rotatable member attached to the fastening means.

3. The method of claim 1, further comprising the step of:
locking the first clip portion to the second clip portion through use of a locking mechanism.

4. The method of claim 1, wherein:
a first strip of compressible material is attached to the bottom side of the first clip portion.

5. The method of claim 4, wherein:
a second strip of compressible material is attached to the top side of the second clip portion.

6. The method of claim 1, wherein:
the two-part clip includes a first tab located at the first end of the first clip portion and a second tab located at the first end of the second clip portion; and
both tabs are positioned and dimensioned to assist in opening and closing the two-part clip.

7. An apparatus secured to a parent to allow skin-on-skin contact with an infant connected to medical tubing or sensors comprising:
a two-part clip capable of maintaining a plurality of medical tubing having a first clip portion and a corresponding second clip portion, the first clip portion having a top side, a bottom side, a first end and a corresponding second end while the corresponding second clip portion also has a top side, a bottom side, a first end and a corresponding second end;
a first spring action hinge connected to the first end of the clip portion as well as the first end of the second clip portion, the first spring action hinge capable of rotating to open and close the first clip portion onto the second clip portion;
a flat panel having a top side and a bottom side, the first spring axis being attached either directly or indirectly onto the top side of the flat panel; and
an adhesive layer attached at the bottom side of the flat panel sufficient to affix the flat panel to the parent.

8. The apparatus of claim 7, further comprising:
a first strip of compressible material attached to the bottom side of the first clip portion.

9. The apparatus of claim 8, further comprising:
a second strip of compressible material attached to the top side of the second clip portion.

10. The apparatus of claim 7, further comprising:
a rotatable member placed between the first spring action hinge and the top side of the flat panel, the rotatable member capable of positioning the two-part clip to maintain one or more medical tubes connected to the infant.

11. The apparatus of claim 7, further comprising:
a locking mechanism attached at the second end of the first clip portion which can position and affix to the second end of the second clip portion to prevent medical tubing or sensors from becoming dislodged from the two-part clip.

12. The apparatus of claim 7, wherein:
the two-part clip includes a first tab located at the first end of the first clip portion and a second tab located at the first end of the second clip portion; and
both tabs are positioned and dimensioned to assist in opening and closing the two-part clip.

13. An apparatus secured to a parent to allow skin-on-skin contact with an infant connected to medical tubing or sensors comprising:
a two-part clip capable of maintaining a plurality of medical tubing having a first clip portion and a corresponding second clip portion, the first clip portion having a top side, a bottom side, a first end and a corresponding second end while the corresponding second clip portion also having a top side, a bottom side, a first end and a second end;
a first spring action hinge connected to the first end of the clip portion as well as the first end of the second clip portion, the first spring action hinge capable of rotating to open and close the first clip portion onto the second clip portion; and
a pair of jaws pivotally mounted on a second spring action hinge sufficient to attach to an article of clothing worn by the parent, the pair of jaws connected either directly or indirectly to the first spring action hinge.

14. The apparatus of claim 13, further comprising:
a first strip of compressible material attached to the bottom side of the first clip portion.

15. The apparatus of claim 14, further comprising:
a second strip of compressible material attached to the top side of the second clip portion.

16. The apparatus of claim 13, further comprising:
a rotatable member placed between the first spring action hinge and the pair of jaws, the rotatable member capable of positioning the two-part clip to maintain one or more medical tubes connected to the infant.

17. The apparatus of claim 13, further comprising:
a locking mechanism attached at the second end of the second clip portion which can position and affix to the second end of the first clip portion to prevent medical tubing from becoming dislodged from the two-part clip.

18. The apparatus of claim 13, wherein:
the two-part clip includes a first tab located at the first end of the first clip portion and a second tab located at the first end of the second clip portion; and
both tabs are positioned and dimensioned to assist in opening and closing the two-part clip.
